(19) United States
(12) Patent Application Publication
(10) Pub. No.: US 2010/0113793 A1
(43) Pub. Date: May 6, 2010

(54) Process for the Preparation of Highly Pure Donepezil

(75) Inventors: Ashvin Kumar Aggarwal, Chandigarh (IN); Chidambaram Vankatesran Srinivasan, Panchkula (IN); Lalit Wadhwa, Panchkula (IN)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(73) Assignee: IND-SWIFT LABORATORIES LIMITED, Mohali, Punjab (IN)

(21) Appl. No.: 12/160,703

(22) PCT Filed: Mar. 20, 2007
(86) PCT No.: PCT/IN2007/000113

§ 371 Date: Jul. 11, 2008

(30) Foreign Application Priority Data
Mar. 20, 2006 (IN) 936/DEL/2006

(51) Int. Cl. C07D 211/06

(52) U.S. Cl. 546/206

(57) ABSTRACT

The present invention relates to the preparation of highly pure donepezil of formula I or salt thereof, by hydrogenating indanonylidenyl compound of formula II, using platinum catalysts.

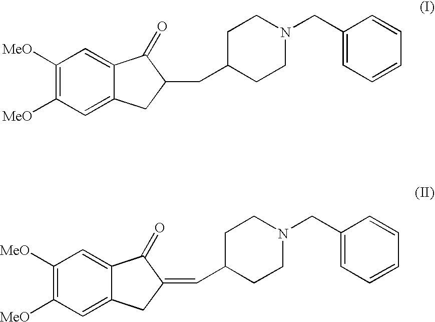

### FIELD OF THE INVENTION

The field of the invention relates to the preparation of highly pure donepezil or its salts in high yields.

### BACKGROUND OF THE INVENTION

Donepezil of formula I, is a cholinesterase inhibitor used in the treatment of mild to moderate cases of Alzheimer's diseases and is chemically known as 1-benzyl-4-[(5,6-dimethoxy-1-indanon)-2-yl] methylpiperidine.

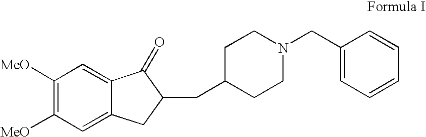

Donepezil was first disclosed in U.S. Pat. No. 4,895,841. Thereafter, several processes for the preparation of donepezil and its salts have published. In U.S. Pat. No. 4,895,841, 5,6-dimethoxy-1-indanone is condensed with 1-benzylpiperidine-4-carboxaldehyde in the presence of strong base, such as lithium diisopropylamide (LDA) to prepare a indanonylidenyl compound of formula II, which on reduction in the presence of palladium on carbon in tetrahydrofuran yield donepezil.

There are significant drawbacks to this method as the catalytic hydrogenation of the indanonylidenyl compound of formula II

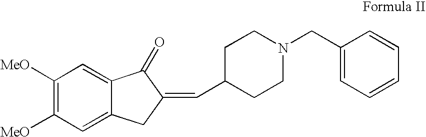

to the corresponding compound of formula I, results in the debenzylation leading in the formation of following impurity of formula III, in substantial amounts,

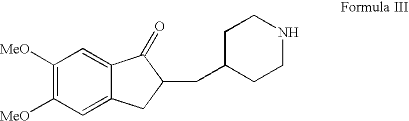

which is difficult to remove. This above impurity is further carried into donepezil hydrochloride. The generation of significant quantity of debenzylated product during hydrogenation makes the process uneconomical. In the exemplified process given in U.S. Pat. No. 4,895,841, column chromatography is utilized to purify the product which is not amenable on industrial scale.

Japanese Patent Application No. JP-A-H11-171861 discloses the preparation of indanoylidenyl compound of formula II by the aldol condensation of 5,6-dimethoxy-1-indanone with 1-benzylpiperidine-4-carboxaldehyde in the presence of alkali-metal alkoxide such as sodium methoxide.

PCT Publication WO 97/22584 discloses preparation of donepezil hydrochloride by reacting pyridine-4-aldehyde with malonic acid. The resulting 3-(pyridin-4-yl)-2-propenoic acid was reduced with rhodium on carbon under hydrogen atmosphere to give 3-(piperidin-4-yl)-2-propionic acid which on reaction with methyl chlorocarbonate gave 3-[N-(methoxycarbonyl)piperidin-4-yl]propionic acid. On reacting 3-[N-(methoxycarbonyl)piperidin-4-yl] propionic acid with oxalyl chloride, methyl 4-(2-chlorocarbonylethyl)piperidin-1-carboxylate is obtained which on reaction with 1,2-dimethoxy benzene in the presence of aluminum chloride afforded methyl 4-[3-(3,4-dimethoxyphenyl)-3-oxopropyl]piperidin-1-carboxylate. On reacting methyl 4-[3-(3,4-dimethoxyphenyl)-3-oxopropyl] piperidin-1-carboxylate with tetramethyl diamino methane, methyl 4-[2-(3,4-dimethoxy benzoyl) allyl] piperidin-1-carboxylate is obtained which on treatment with sulphuric acid gave methyl 4-(5,6-dimethoxy-1-indanon-2-yl methyl)piperidin-1-carboxylate. On decarboxylation of methyl 4-(5,6-dimethoxy-1-indanon-2-yl methyl)piperidin-1-carboxylate gave 5,6-dimethoxy-2-(piperidin-4-yl methyl)-1-indanone which on treatment with benzyl bromide afforded donepezil hydrochloride.

U.S. Pat. No. 5,606,064 discloses another process for the preparation of donepezil, which involves reacting 5,6-dimethoxy-1-indanone and pyridine-4-carboxaldehyde to yield 5,6 dimethoxy-2-(pyridin-4-yl)-methylene-indan-1-one, which upon treatment with benzyl bromide followed by reduction yields the required compound.

U.S. Pat. No. 6,492,522 discloses an alternative process for the preparation of donepezil hydrochloride which comprises the steps of carrying out the intramolecular cyclization of N-benzyl-2-(3,4-dimethoxybenzyl)-3-(4-piperidine)propionic acid of formula IV,

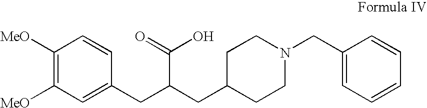

to yield crude donepezil and isolating the pure donepezil by chromatography or crystallization.

U.S. Pat. No. 6,252,081 discloses an additional process for production of donepezil hydrochloride which involves the reaction of donepezil intermediate of formula V,

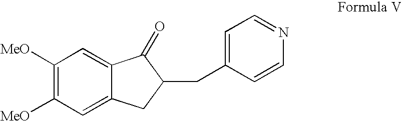

with halogenated benzyl to obtain a quaternary ammonium salt, hydrogenation of the quaternary ammonium salt to produce donepezil base, followed by hydrohalogenation to produce donepezil hydrochloride.

The prior art processes employs reaction of 1-benzylpiperidine-4-carboxaldehyde with 5,6-dimethoxy-1-indanone involves use of lithium diisopropylamide. Lithium diisopropylamide is toxic and needs to be carefully handled. Use of oxalyl chloride chemistry is difficult for scale up. Besides the oxalyl reaction also involves many protection deprotection chemistry and the overall yield is very low. Raw materials like methyl chlorocarbonate or tetramethyl diaminomethyl are expensive and difficult to source commercially.

In view of the above, the prior art processes are time consuming and difficult to carry out as they involve many steps. Most of the prior art approaches are not suitable from commercial point of view because the desired product is not obtained in high purity and requires purification by tedious and cumbersome purification processes. The inventors have observed that during the condensation reaction of 5,6-dimethoxy-1-indanone of formula-VI,

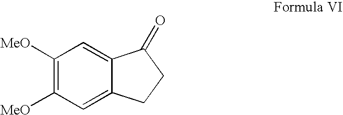

with 1-benzylpiperidine-4-carboxaldehyde of formula-VII,

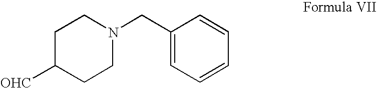

for the preparation of indanonylidenyl compound of formula II

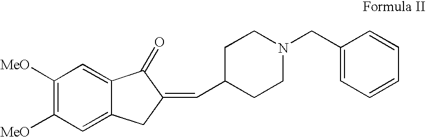

formation of a hydroxyl impurity of compound of formula VIII,

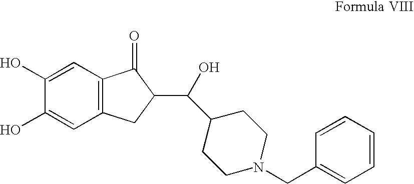

occurs upto 20%. The amount of impurity formation varies depending upon reaction conditions, amount of solvent, nature of solvent used. It is advantageous to remove hydroxyl impurity at this stage; otherwise it converts to indanonylidenyl compound of formula II during preparation of donepezil hydrochloride in the presence of hydrochloric acid, hence difficult to remove. Further hydrogenation is required to reduce the double bond, which is an additional step leading to increase in cost As discussed above, the inventors have further found that during hydrogenation of indanonylidenyl compound of formula II in the presence of palladium catalyst debenzylation occurs to the extent of 10%. Once the debenzylated product is formed in reaction mixture it is difficult to remove. An additional step of benzylation is required to convert the debenzylated product to desired compound. To achieve a high efficiency of the reaction for industrial synthesis of donepezil, it is necessary to minimize the formation of the impurities and improve the yields.

Thus, the present invention provides an efficient and industrially advantageous process for the preparation of highly pure donepezil hydrochloride in high yields.

### SUMMARY OF THE INVENTION

Accordingly, in one aspect, present invention provides an improved process for the preparation of highly pure donepezil of formula I

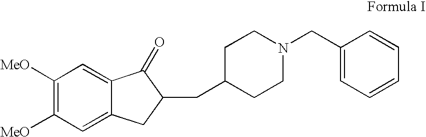

or its salts which comprises:
hydrogenating indanonylidenyl compound of formula II;

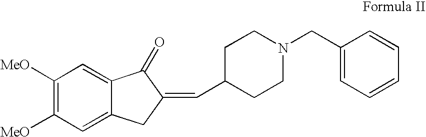

using platinum catalyst in an organic solvent at a temperature of 0-50° C.;
preparing donepezil base and converting to pharmaceutically acceptable salts.

In another aspect, present invention provides a process for the preparation of indanonylidenyl compound of formula II which comprises:

condensing 5,6-dimethoxy-1-indanone of formula-VI,

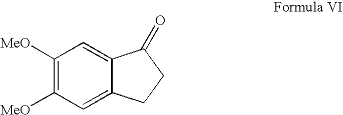

and 1-benzylpiperidine-4-carboxaldehyde of formula-VII,

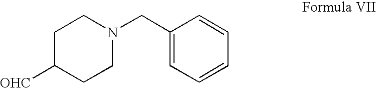

in the presence of alkali metal alkoxide in methanol as solvent.

In another aspect, present invention provides a process for the purification of indanonylidenyl compound of formula II to remove hydroxy impurity of formula VIII,

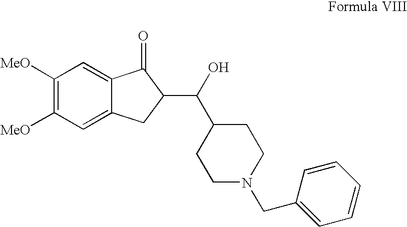

by converting compound of formula VIII into indanonylidenyl compound of formula II by using acid base treatment or protection/deprotection of hydroxy group.

### DETAILED DESCRIPTION OF THE INVENTION

The instant invention relates to an improved, efficient and industrially advantageous process for the preparation of highly pure donepezil of formula-I or salt thereof.

According to an embodiment, there is provided a process for the preparation of indanonylidenyl compound of formula-II in high purity and better yields by the condensation of 5,6-dimethoxy-1-indanone of formula-VI,

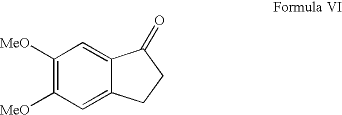

and 1-benzylpiperidine-4-carboxaldehyde of formula-VII,

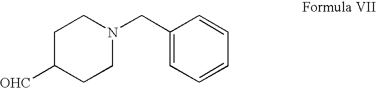

in the presence of alkali metal alkoxide in a solvent. Specifically the reaction is conducted at 30-65° C. in methanol and it takes about 2-7 hours for completion of reaction. The indanonylidenyl compound of formula-II may be further purified by several ways such as by using acid base treatment; by protecting deprotecting hydroxyl group, to remove the hydroxyl impurity of formula VIII, by converting

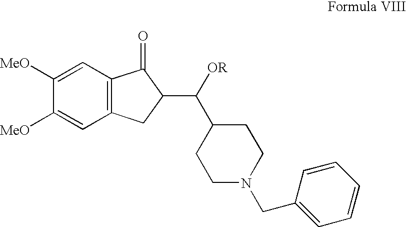

compound of formula VIII into indanonylidenyl compound of formula II.

Generally, the crude indanonylidenyl compound of formula-II, having hydroxy impurity is treated with organic or inorganic acid in the presence of solvent such as ethers, alcohols, ketones, hydrocarbons, polar aprotic solvents. Specifically the compound of formula-II is treated with methanolic-hydrochloric acid in methanol. After work up, pure hydrochloride of indanonylidenyl compound of formula-II is isolated, which is then basified to get pure indanonylideny compound of formula II. Alternatively the hydroxy impurity of formula VIII can be removed by converting hydroxy compound of formula VIII to compound of formula IX by protecting the hydroxyl group with suitable hydroxyl protecting group, and in situ converting compound of formula IX to compound of formula II by dehydration using conventional methods. The protecting hydroxy group for the compound of formula IX can be selected from acetates, benzoates, sulfonates and the like which are prepared by using suitable reagents known in prior art.

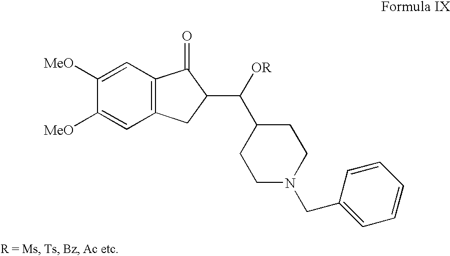

In one specific embodiment of the present invention sulfonates derivatives of formula IX are prepared. Sulfonates can be methanesulfonate, 4-toluenesulfonate, benzenesulfonate and the like. The compound of formula VIII may be treated with sulfonyl chloride in the presence of an organic base and a solvent. The solvent comprises one or more of ethers, alcohols, chlorinated hydrocarbons, esters, ketones, hydrocarbons, polar aprotic solvents. It is advantageous to remove impurity of formula VIII before carrying out hydrogenation; otherwise it would carry forward. During hydrochloride preparation of donepezil it is converting to compound of formula II on dehydration, in the presence of hydrochloric acid. It is very difficult to remove indanonylidenyl compound of formula-II from donepezil hydrochloride and further hydrogenation is required to reduce the double bond, which is an additional step leading to increase in cost.

After purification, the indanonylidenyl compound of formula II is isolated in high purity greater than 98.5% preferably greater than 99.8%.

According to another embodiment, there is provided a process for the preparation of Gpezil of formula I which includes hydrogenating indanonylidenyl compound of formula-II, in the presence of platinum catalyst to produce and isolating the substantially pure donepezil or a salt thereof. Specifically hydrogenation of indanonylidenyl compound of formula II is carried out using platinum catalyst in an organic solvent at 0-50° C. preferably at 25-40° C. under 1-5 atmospheric pressure preferably at 4 atmospheric pressure. The organic solvent can be selected from solvent comprises one or more of ethers, alcohols, chlorinated hydrocarbons, esters, ketones, hydrocarbons, polar aprotic solvents, water and mixtures thereof and preferably ethyl acetate is used. We have carried out same reactions using palladium catalyst also and found that using palladium catalyst, impurity of formula III is formed in substantial amount depending upon reaction time. The examples have been provided as comparative examples. Platinum catalysts are mild in nature therefore debenzylation is not observed using platinum catalysts. Platinum catalyst can be selected from platinum oxide, platinum on carbon and preferably platinum on carbon is used. The reaction can be accomplished at 0-50° C. and preferably at 25-35° C. The progress of the reaction is monitored by high performance liquid chromatography (HPLC), and the reaction is stirred till the starting material i.e. compound of formula II remains less than 0.15%, the reaction mixture is filtered to recover the catalyst. The filtrate is concentrated and crude donepezil is dissolved in organic solvent. The organic solvent is selected from methylene chloride, ethyl acetate and preferably methylene chloride is used. The reaction mixture is treated with a solution of hydrochloric acid in ethyl acetate to obtain donepezil hydrochloride.

The resulting mixture is concentrated and the product obtained is recrystallized from methanol and isopropyl ether to obtain highly pure donepezil hydrochloride having purity greater than 99.5% preferably greater than 99.8%.

Major advantages realized in the present invention are high purity and high yields. The formation of impurity of formula-III has been restricted by making use of platinum catalyst. This avoids the use of tedious and cumbersome technique i.e. chromatographic purification, and additional step of benzylation of compound of formula-III. The hydroxy impurity of formula VIII is removed by acid base treatment or protection/deprotection technique.

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### EXAMPLES

### Preparation of 1-Benzyl-4-[5,6-dimethoxy-1-indanon)-2-ylidenyl]methyl piperidine

Sodium hydroxide (1.46 g) was added to methanol (150 ml) followed by addition of 5,6-dimethoxy-1-indanone (5 g) and 1-benzylpiperidene-4-carboxaldehyde (6.8 g) at 20-30° C. The temperature of the reaction mass was gradually raised to 62-66° C. and was stirred for 6 hours. The reaction mass was concentrated under vacuum and ⅔ volume of the reaction mass was recovered. The remaining slurry was filtered and the product was washed with water followed by methanol to give 6.9 g of 1-benzyl-4-[5,6-dimethoxy-1-indanon)-2-ylidenyl] methyl piperidine (yield=70%), having purity 98.5% by HPLC.

### Preparation of Benzyl-4-[(5,6-dimethoxy-1-indanon)-2-ylidenyl] methyl piperidine

A solution 9.8 g of sodium methoxide in 60 ml of methanol was added to a stirred solution of 25 g of 5,6-dimethoxy-1-indanone and 36.75 g 1-benzyl piperidene-4-carboxaldehyde in methanol (690 ml) at 20-30° C. The temperature of the reaction mass was gradually raised to 60-65° C. and stirred for 2 hours. The precipitated solid was filtered and the product was washed with water followed by methanol to give crude product which was converted to its hydrochloride salt using methanol. The hydrochloride salt was basified using sodium hydroxide to obtain 26.9 g of 1-benzyl-4-[5,6-dimethoxy-1-indanon)-2-ylidenyl] methyl piperidine (yield=60%) having purity 99.92% by HPLC.

### Purification of 1-Benzyl-4-[(5,6-dimethoxy-1-indanone)-2-ylidenyl] methyl piperidine

To a stirred solution of 1-benzyl-4-[5,6-dimethoxy-1-indanon)-2-ylidenyl] methyl piperidine (10 g, purity 93.38%)) in methylene chloride (200 ml) was added methane sulfonyl chloride (0.2 ml) and triethylamine at 25-30° C. The reaction mixture was stirred at 20-30° C. and after completion of reaction, the reaction mass was quenched with DM water. The organic layer was washed with aqueous sodium bicarbonate solution followed by concentration in vacuum to obtain the product which was filtered with methanol to obtain 7.7 gm of 1-benzyl-4-[(5,6-dimethoxy-1-indanone)-2-ylidenyl] methylpiperidine having a purity of 98.71% by HPLC.

### Purification of Benzyl-4-[(5,6-dimethoxy-1-indanon)-2-ylidenyl] methyl piperidine

1-Benzyl-4-[5,6-dimethoxy-1-indanon)-2-ylidenyl] methyl piperidine (80 g, purity 98.85%) was taken in methanol (400 ml) followed by addition of methanolic solution of hydrogen chloride (48 ml) at 10-15° C. The product was filtered followed by addition in the mixture of DM water (400 ml) and methylene chloride (640 ml), the Ph of the mass was adjusted to ˜9 with 10% caustic solution at 20-30° C. The organic layer was separated and concentration in vacuum to obtain the product which was filtered, washed with methanol to obtain 67 gm of 1-benzyl-4-[(5,6-dimethoxy-1-indanone)-2-ylidenyl] methylpiperidine having a purity 99.67% by HPLC.

### Preparation of 1-Benzyl-4-[(5,6-dimethoxy-1-indanon)-2-yl] methylpiperidine hydrochloride (Donepezil Hydrochloride)

1-Benzyl-4-[5,6-dimethoxy-1-indanon)-2-ylidenyl] methyl piperidine (10 g) was taken in ethyl acetate (200 ml) followed by addition of 5% platinum-carbon (2 g). The reaction mixture was hydrogenated at temperature 20-30° C. and two atmospheric pressure for 2 hours. After completion of reaction, the catalyst was filtered off and the filtrate was concentrated in vacuum. The residue was dissolved in methylene chloride and a solution of hydrochloric acid in ethyl acetate was added to the resulting solution, followed by concentration in vacuum to obtain a crystal, which was recrystallized from methanol/isopropyl ether to obtain 10.6 g of 1-benzyl-4-[(5,6-dimethoxy-1-indanone)-2-yl]methylpiperidine hydrochloride having a purity of 99.55% by HPLC.

### Preparation of 1-Benzyl-4-[(5,6-dimethoxy-1-indanon)-2-yl] methylpiperidine hydrochloride (Donepezil Hydrochloride)

1-Benzyl-4-[5,6-dimethoxy-1-indanon)-2-ylidenyl]methyl piperidine (10 g) was hydrogenated in ethyl acetate (100 ml) for 2 hours in presence of 5% platinum-carbon (2 g) at 4 atmospheric pressure. The reaction mixture was filtered and concentrated under vacuum to give residue. The residue was dissolved in methylene chloride and further acidified with a solution ethyl acetate hydrochloric acid at 0-10° C. followed by concentration in vacuum to obtain a solid which was recrystallized from methanol and isopropyl ether to obtain 7.4 g of 1-benzyl-4-[(5,6-dimethoxy-1-indanon)-2-yl] methylpiperidine hydrochloride (yield=67%) having a purity of 99.93% by HPLC

### Preparation of 1-Benzyl-4-[(5,6-dimethoxy-1-indanon)-2-yl] methylpiperidine hydrochloride (Donepezil Hydrochloride)

1-Benzyl-4-[5,6-dimethoxy-1-indanon)-2-ylidenyl]methyl piperidine (8 g) was taken in ethyl acetate (160 ml) followed by addition of 5% platinum-carbon (1.6 g). The reaction mass was hydrogenated at 20-30° C. under 2 atmospheric pressure for 2 hours and the progress of the reaction was monitored by HPLC analysis (debenzlated impurity is <0.25%). The catalyst was filtered off and the filtrate was concentrated in vacuum. The residue was dissolved in methylene chloride and a solution of hydrochloric acid in ethyl acetate was added to the resulting solution, followed by concentration in vacuum to obtain solid, which was recrystallized from methanol/isopropyl ether to obtain 6.8 g of 1-benzyl-4-[(5,6-dimethoxy-1-indanon)-2-yl]methylpiperidine hydrochloride having purity of 99.56% by HPLC.

### Reference Examples

### Preparation of 1-Benzyl-4-[(5,6-dimethoxy-1-indanon)-2-yl] methylpiperidine hydrochloride (Donepezil Hydrochloride)

1-Benzyl-4-[5,6-dimethoxy-1-indanon)-2-ylidenyl] methyl piperidine was taken in tetrahydrofuran (200 ml) followed by addition of palladium-carbon (1 g). The mixture was hydrogenated at 20-30° C. under 1.5 atmospheric pressure for 8 hours, monitored by HPLC analysis and analysis shows ˜38.5% of debenzylated impurity in the reaction mass.

### Preparation of 1-Benzyl-4-[(5,6-dimethoxy-1-indanon)-2-yl] methylpiperidine hydrochloride (Donepezil Hydrochloride)

1-Benzyl-4-[5,6-dimethoxy-1-indanon)-2-ylidenyl] methyl piperidine was taken in 200 ml THF followed by addition of 10% palladium-carbon (0.5 g). The mixture was hydrogenated at 20-30° C. under 1.5 atmospheric pressure for 8 hour and the progress of the reaction was monitored by HPLC analysis (the analysis showed ˜22.4% of debenzylated impurity in the reaction mass after 4 hours and ˜38% after 8 hours).

### Preparation of 1-Benzyl-4-[(5,6-dimethoxy-1-indanon)-2-yl] methylpiperidine hydrochloride (Donepezil Hydrochloride)

1-Benzyl-4-[5,6-dimethoxy-1-indanon)-2-ylidenyl] methyl piperidine (5 g) was taken in 100 ml of THF followed by addition of 10% palladium-carbon (0.5 g). The reaction mixture was hydrogenated at temperature of 20-30° C. at 0.3 atmospheric pressure for 2 hour and the progress of the reaction was monitored by HPLC analysis (the HPLC analysis showed the debenzylated impurity >9%).

1. A process for the preparation of highly pure Donepezil of formula I 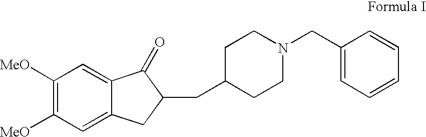
or its salts which comprises:
hydrogenating indanonylidenyl compound of formula II 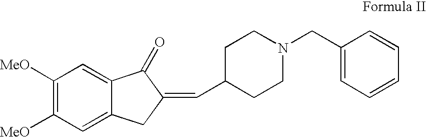
using platinum catalyst in an organic solvent at a temperature of 0-50° C., preparing donepezil base and converting to pharmaceutical acceptable salts.

2. The process according to claim 1, wherein platinum catalyst is preferably platinum on carbon.

3. The process of claim 1, wherein the organic solvent comprises one or more of ethers, alcohols, chlorinated hydrocarbons, esters, ketones, hydrocarbons, polar aprotic solvents, water and mixtures thereof.

4. The process according to claim 1, for the preparation of indanonylidenyl compound of formula II 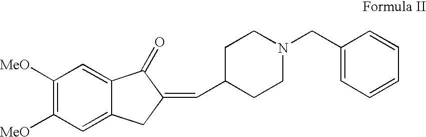
which comprises,
condensation of 5,6-dimethoxy-1-indanone of formula VI, 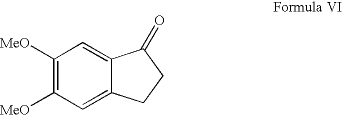
with 1-benzylpiperidene-4-carboxaldehyde of formula VII, 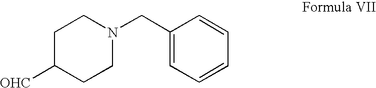
in the presence of alkali metal hydroxide in a solvent such as one or more of ethers, alcohols, chlorinated hydrocarbons, esters, ketones, hydrocarbons, polar aprotic solvents, water and mixtures thereof.

5. The process according to claim 1, wherein alkali metal hydroxide is selected from sodium hydroxide, potassium hydroxide.

6. The process according to claim 1, wherein the alcohol comprises one or more of methanol, ethanol, propanol, isopropanol and butanol.

7. A process for the purification of indanonylidenyl compound of formula II 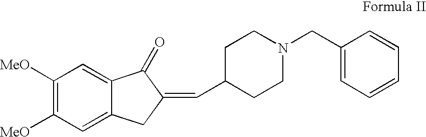
to remove the hydroxy impurity of formula VIII, 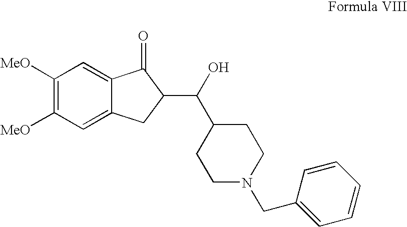
by converting compound of formula VIII into indanonylidenyl compound of formula II in presence of organic acid/inorganic acid in the presence of solvent such as ethers, alcohols, ketones, hydrocarbons, polar aprotic solvents.

8. The process of claim 7, wherein the acid is hydrochloric acid.

9. A process for the purification of indanonylidenyl compound of formula II 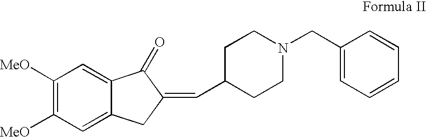
to remove the hydroxy impurity of formula VIII, 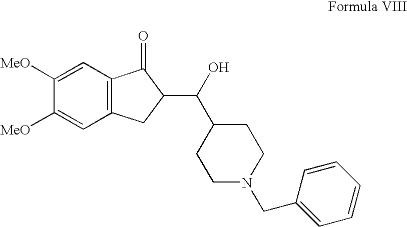
by converting compound of formula VIII into indanonylidenyl compound of formula II, in the presence of suitable reagent such as sulfonates and organic base in a solvent such as ethers, chlorinated hydrocarbons, esters, ketones, hydrocarbons, polar aprotic solvents.

10. The process of claim 9, wherein the organic base is triethylamine, diisopropyl ethyl amine, pyridine, piperidine preferably triethyl amine.
